(19) United States
(12) Patent Application Publication
(10) Pub. No.: US 2022/0160806 A1
(43) Pub. Date: May 26, 2022

(54) CONDOM HAVING ANTI-BACTERIAL AND ANTI-INFLAMMATORY FUNCTION

(71) Applicant: GUSTAR CO., LTD., Gyeonggi-do (KR)

(72) Inventor: Sujin YANG, Seoul (KR)

(21) Appl. No.: 17/440,875

(22) PCT Filed: Feb. 24, 2020
(86) PCT No.: PCT/KR2020/002639

§ 371 Date: Sep. 20, 2021

(30) Foreign Application Priority Data
Mar. 20, 2019 (KR) 10-2019-0031746

(51) Int. Cl. A61K 36/73; A61F 6/04; A61K 36/65; A61K 36/539; A61K 31/352; A61K 9/00

(52) U.S. Cl. CPC A61K 36/73; A61F 6/04; A61K 9/0034; A61K 36/539; A61K 31/352; A61K 36/65

(57) ABSTRACT

A condom according to an embodiment of the present disclosure may be produced at low cost and has antibacterial and anti-inflammatory activity. The condom may include a condom body, and a lubricant layer formed on the outer surface of the condom body by applying a lubricant solution comprising a mixture of a lubricant and a composition for antibacterial and anti-inflammatory activities to the outer surface. The composition contains, as an active ingredient, a combination of four components consisting of a fisetin compound, a Rose damascene extract, a Paeonia suffruticosa Andr. extract, and a Scutellaria baicalensis extract. As the lubricant solution applied to the outer surface of the condom body is produced by mixing the lubricant with the composition for antibacterial and anti-inflammatory activities, the condom may exhibit antibacterial and anti-inflammatory activities in a woman's vagina.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2020/002639, filed Feb. 24, 2020, which claims priority to the benefit of Korean Patent Application No. 10-2019-0031746 filed in the Korean Intellectual Property Office on Mar. 20, 2019, the entire contents of which are incorporated herein by reference.

### BACKGROUND

### 1. Technical Field

The present invention relates to a condom, and more particularly, to a condom having antibacterial and anti-inflammatory activities.

### 2. Background Art

A condom is a contraceptive device that can prevent unwanted pregnancy by preventing sperm from entering the vagina during sexual intercourse. A conventional common is made of a thin rubber material so that it may easily accommodate a male penis, and a lubricant is applied to the outer surface of the condom so that the condom can be easily inserted into a woman's vagina. However, the conventional condom does not have antibacterial and anti-inflammatory activities.

Meanwhile, in recent years, condoms having antibacterial and anti-inflammatory activities have been developed. For example, Korean Utility Model Registration No. 20-0207212 (published on Dec. 15, 2000) discloses a condom containing gold and silver. That is, the condom disclosed therein contains a silver foil or gold and silver together with a lubricant component on the inside or outside thereof so that it may have an effect of preventing bacterial infection as well as a cell regeneration effect or a disinfectant effect.

In addition, Korean Patent No. 10-0884241 (published on Feb. 17, 2009) discloses a condom coated with nano-silver powder which is a material having a strong antibacterial and disinfectant activities.

However, the above-described condoms having antibacterial activity are produced using gold or silver as an active material, and thus have a problem in that the production cost thereof increases.

### SUMMARY

Therefore, the present invention is intended to solve the above-described problem, and the problem to be solved by the present invention is to provide a condom which is produced at low cost and has antibacterial and anti-inflammatory activities.

A condom according to one embodiment of the present invention comprises: a condom body; and a lubricant layer formed on the outer surface of the condom body by applying a lubricant solution comprising a mixture of a lubricant and a composition for antibacterial and anti-inflammatory activities to the outer surface. The composition contains, as an active ingredient, a combination of four components consisting of a fisetin compound, a Rose damascene extract, a Paeonia suffruticosa Andr. extract, and a Scutellaria baicalensis extract.

The fisetin compound, a kind of flavonoid, may be a compound represented by a molecular formula of C₁₅H₁₀O₆.

The fisetin compound may be a compound represented by the following formula:

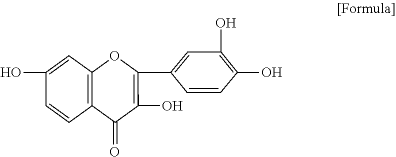 [SMILES: O=c1c(O)c(-c2ccc(O)c(O)c2)oc2cc(O)ccc12]

The Rose damascene extract may be a substance obtained by extraction from Rose damascene which is a small shrub belonging to the family Rosaceae of the order Rosales of Dicotyledoneae; the Paeonia suffruticosa Andr. extract may be a substance obtained by extraction from Paeonia suffruticosa Andr. which is a plant belonging to the family Ranunculaceae of Dicotyledoneae; and the Scutellaria baicalensis extract may be a substance obtained by extraction from Scutellaria baicalensis which is a perennial plant belonging to the family Labiatae of the order Tubiflorae of Dicotyledoneae.

The composition may be produced by mixing the fisetin compound, the Rose damascene extract, the Paeonia suffruticosa Andr. extract and the Scutellaria baicalensis extract together at a weight ratio of 1:0.5 to 1.5:8 to 12:8 to 12.

The composition may be produced by mixing the fisetin compound, the Rose damascene extract, the Paeonia suffruticosa Andr. extract and the Scutellaria baicalensis extract together at a weight ratio of 1:1:10:10.

The lubricant solution may be prepared by mixing the lubricant with 0.001 to 10 wt % of the composition.

According to the condom of the present invention, as the lubricant solution applied to the outer surface of the condom body is produced by mixing the lubricant with the composition for antibacterial and anti-inflammatory activities, the condom may exhibit antibacterial and anti-inflammatory activities in a woman's vagina.

In addition, as the composition contains the extracts produced by extraction from natural plant materials, it is harmless to the human body and may have a function of effectively preventing infection by harmful microorganisms, and the condom may be produced at low cost compared to a conventional condom which is produced using gold or silver as a material.

### BRIEF DESCRIPTION OF THE DRAWINGS

FIG. 1 is a sectional view illustrating a condom according to one embodiment of the present invention.

FIG. 2 is an enlarged sectional view of portion “A” of FIG. 1.

### DETAILED DESCRIPTION

Although the present invention may be modified variously and may have several embodiments, specific embodiments are illustrated in the accompanying drawings and will be described in detail in the detailed description.

However, it is to be understood that the present invention is not limited to the specific embodiments and includes all the modifications, equivalents and substitutions included in the spirit and scope of the present invention. Terms, such as “first” and “second,” may be used to describe various components, but the components are not limited by the terms. The terms are used merely for the purpose of distinguishing a component from other components. For example, the first component may be designated as the second component without departing from the scope of the present invention, and, similarly, the second component may also be designated as the first component.

The terms used in the present specification are used only to describe specific embodiments, and are not intended to limit the present invention. Singular expressions include plural expressions unless the context clearly indicates otherwise. In the present specification, it should be understood that terms such as “comprise” and “have” are intended to denote the existence of mentioned characteristics, numbers, steps, operations, components, parts, or combinations thereof, but do not exclude the probability of existence or addition of one or more other characteristics, numbers, steps, operations, components, parts, or combinations thereof.

Hereinafter, preferred embodiments of the present invention will be described in more detail with reference to the accompanying drawings.

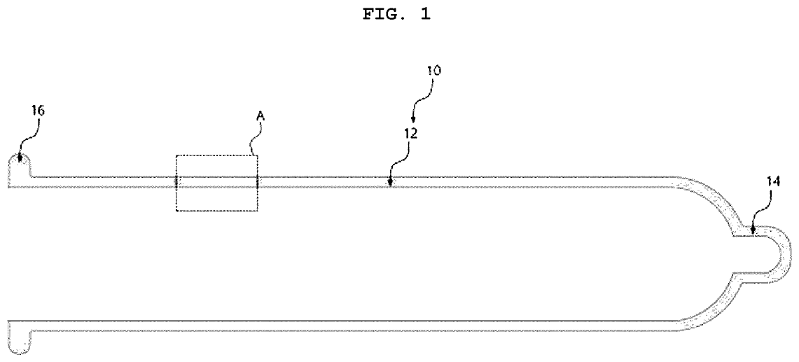

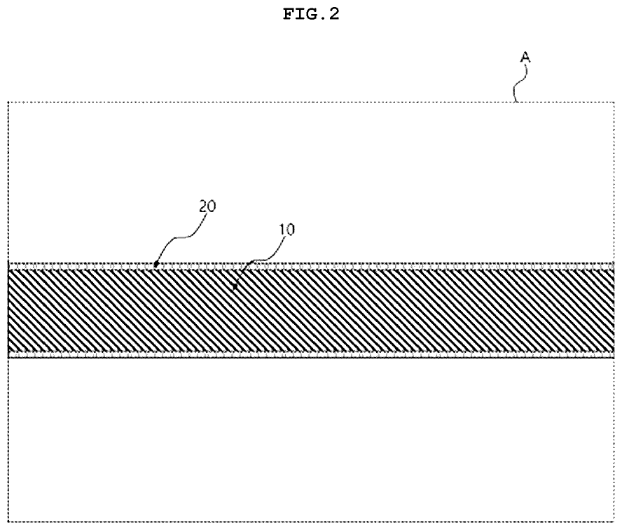

FIG. 1 is a sectional view illustrating a condom according to one embodiment of the present invention, and FIG. 2 is an enlarged sectional view of portion “A” of FIG. 1.

Referring to 1 and 2, a condom according to one embodiment of the present invention is a contraceptive device into which the male penis may be easily inserted and which may prevent unwanted pregnancy by preventing sperm from entering the female vagina during sexual intercourse. The condom may comprise a condom body 10 for accommodating a male penis, and a lubricant layer 20 formed on the outer surface of the condom body 10.

The condom body 10 may comprise: a body portion 12 into which a male penis is inserted and accommodated; a front end protrusion 14 protruding from the center of the front end of the body portion 12; and a side protrusion 16 formed along the edge of the rear end of the body portion 12. The condom body 10 may be made of an elastic rubber material, for example, natural rubber latex.

The lubricant layer 20 may comprise a lubricant solution existing in, for example, a liquid or gel state. In this case, the lubricant solution may be applied to the outer surface of the condom body 10 to form the lubricant layer 20. When the lubricant layer 20 is formed on the outer surface of the condom body 10 as described above, the condom body 10 into which a male penis has been inserted may be easily inserted into a female vagina.

In addition, the lubricating layer 20 may also be formed on the inner surface of the condom body 10. That is, the lubricant solution may also be applied to the inner surface of the condom body 10. When the lubricant layer 20 is also formed on the inner surface of the condom body 10 as described above, a male penis may be easily inserted into the condom body 10.

Meanwhile, a certain amount of the lubricant solution may exist in the inner space formed in the side projection 16. In this case, when the condom body 10 into which a male penis has been inserted is inserted into and moves forward and backward in a female vagina, the lubricant solution present in the inner space of the side protrusion 16 may be discharged to the outer surface of the condom body 10 by pressure and impact.

In this embodiment, the lubricant solution forming the lubricant layer 20 may be a mixture of a lubricant present in a solid or gel state and the composition for antibacterial and anti-inflammatory activities.

In this embodiment, the composition contained in the lubricant solution may contain, as an active ingredient, a combination of four components consisting of a fisetin compound, a Rose damascene extract, a Paeonia suffruticosa Andr. extract, and a Scutellaria baicalensis extract.

The fisetin compound, a kind of flavonoid, may have a molecular formula of C₁₅H₁₀O₆, may be represented by the following Formula 1, and can be found mainly in the heartwoods of Anacardiaceae plants in nature.

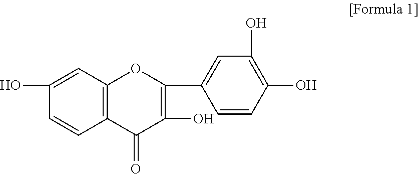 [SMILES: O=c1c(O)c(-c2ccc(O)c(O)c2)oc2cc(O)ccc12]

The Rose damascene extract may be a substance obtained by extraction from Rose damascene which is a small shrub belonging to the family Rosaceae of the order Rosales of Dicotyledoneae; the Paeonia suffruticosa Andr. extract may be a substance obtained by extraction from Paeonia suffruticosa Andr. which is a plant belonging to the family Ranunculaceae of Dicotyledoneae; and the Scutellaria baicalensis extract may be a substance obtained by extraction from Scutellaria baicalensis which is a perennial plant belonging to the family Labiatae of the order Tubiflorae of Dicotyledoneae.

The composition may be produced by mixing the fisetin compound, the Rose damascene extract, the Paeonia suffruticosa Andr. extract and the Scutellaria baicalensis extract together at a weight ratio of 1:0.5 to 1.5:8 to 12:8 to 12 as determined by checkerboard assay. More specifically, the composition contained in the lubricant solution may be produced by mixing the fisetin compound, the Rose damascene extract, the Paeonia suffruticosa Andr. extract and the Scutellaria baicalensis extract together at a weight ratio of 1:1:10:10.

Meanwhile, the composition may have very excellent antibacterial activity. For example, the composition may have excellent antifungal activity against fungi of the genus Candida, more specifically, Candida albicans. In addition, the composition may have excellent antibacterial activity against gram-negative strains of the genus Escherichia, more specifically Escherichia coli, and gram-positive strains of the genus Staphylococcus, more specifically, Staphylococcus aureus.

In addition, as the composition contains the extracts produced by extraction from natural plant materials, it is harmless to the human body and may have a function of effectively preventing infection by harmful microorganisms.

In this embodiment, the lubricant solution may be prepared by mixing the lubricant with 0.001 to 10 wt % of the composition.

As described above, as the lubricant solution which is applied to the outer surface of the condom body 10 is produced by mixing the lubricant with the composition for antibacterial and anti-inflammatory activities, the condom may exhibit antibacterial and anti-inflammatory activities in a woman's vagina.

In addition, as the composition contains the extracts produced by extraction from natural plant materials, it is harmless to the human body and may have a function of effectively preventing infection by harmful microorganisms, and the condom may be produced at low cost compared to a conventional condom which is produced using gold or silver as a material.

Although the present invention has been described above in detail with reference to preferred embodiments of the present invention, those of ordinary skill in the art will appreciate that various modifications and alterations of the present invention are possible without departing from the spirit and scope of the present invention as defined in the appended claims.

1. A condom comprising:
a condom body; and
a lubricant layer formed on an outer surface of the condom body by applying a lubricant solution comprising a mixture of a lubricant and a composition for antibacterial and anti-inflammatory activities to the outer surface, the composition comprising, as an active ingredient, a combination comprising a fisetin compound, a Rose damascene extract, a Paeonia suffruticosa Andr. extract, and a Scutellaria baicalensis extract.

2. The condom of claim 1, wherein the fisetin compound is a kind of flavonoid and is a compound represented by a molecular formula of C₁₅H₁₀O₆.

3. The condom of claim 2, wherein the fisetin compound is a compound represented by the following formula:

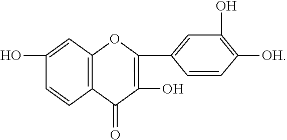 [SMILES: O=c1c(O)c(-c2ccc(O)c(O)c2)oc2cc(O)ccc12]

4. The condom of claim 2, wherein the Rose damascene extract is a substance obtained by extraction from Rose damascene which is a small shrub belonging to the family Rosaceae of the order Rosales of Dicotyledoneae;
the Paeonia suffruticosa Andr. extract is a substance obtained by extraction from Paeonia suffruticosa Andr. which is a plant belonging to the family Ranunculaceae of Dicotyledoneae; and
the Scutellaria baicalensis extract is a substance obtained by extraction from Scutellaria baicalensis which is a perennial plant belonging to the family Labiatae of the order Tubiflorae of Dicotyledoneae.

5. The condom of claim 4, wherein the composition is produced by mixing the fisetin compound, the Rose damascene extract, the Paeonia suffruticosa Andr. extract and the Scutellaria baicalensis extract together at a weight ratio of 1:0.5 to 1.5:8 to 12:8 to 12.

6. The condom of claim 5, wherein the composition is produced by mixing the fisetin compound, the Rose damascene extract, the Paeonia suffruticosa Andr. extract and the Scutellaria baicalensis extract together at a weight ratio of 1:1:10:10.

7. The condom of claim 1, wherein the lubricant solution is prepared by mixing the lubricant with 0.001 to 10 wt % of the composition.
